(19) United States
(12) Patent Application Publication
(10) Pub. No.: US 2021/0219955 A1
(43) Pub. Date: Jul. 22, 2021

(54) Endoscopic Ultrasound Probe And Sheath For Such A Probe

(71) Applicant: Universite Grenoble Alpes, Saint Martin D'heres (FR)

(72) Inventors: Guillaume Custillon, Echirolles (FR); Nicolas Dubois, Montbonnot Saint Martin (FR)

(73) Assignee: Universite Grenoble Alpes, Saint Martin D'heres (FR)

(21) Appl. No.: 15/734,071

(22) PCT Filed: Jun. 3, 2019
(86) PCT No.: PCT/FR2019/051293

§ 371 Date: Dec. 1, 2020

(30) Foreign Application Priority Data
Jun. 1, 2018 (FR) 1854803

(51) Int. Cl. A61B 8/00; A61B 8/12; A61B 8/08

(52) U.S. Cl. CPC A61B 8/4422; A61B 8/12; A61B 8/5223; A61B 8/445; A61B 8/0858

(57) ABSTRACT

The invention relates to a sheath (10) for an endoscopic ultrasound probe, comprising:
a first flexible portion (10a) suited to surrounding a catheter (4) of the ultrasound probe, made of a material suited to allowing ultrasounds to pass, having a closed distal end,
a second portion (10b) suited to surrounding a handle (2) and a cable (3) for electrically connecting the probe to an ultrasound station, and
a fitting (11) arranged between the first and the second portion (10a, 10b), said fitting (11) being suited to being removably attached to the handle (2).

### FIELD OF THE INVENTION

The present invention relates to an endoscopic ultrasound probe, suited to being used within the context of an arthroscopy, notably for minimally invasive intraoperative intra-articular imaging, as well as a sterile sheath for such a probe.

### PRIOR ART

Medical imaging groups together means for acquiring and rendering images from different physical phenomena: magnetic resonance, reflection of ultrasonic waves, radioactivity, absorption of X-rays, etc. These technologies make it possible to view the physiology or the metabolism of the human body, to establish a precise diagnosis and to orientate therapeutic choices. However, they represent an imperfect imaging panoply: some information remains inaccessible or not very user-friendly (notably not being in real time), and irradiations are recognised as a major problem for patients and medical staff.

Only certain technologies are available in the operating theatre depending on the type of surgery. Orthopaedics requires the identification of bone structures and thus mainly resorts to the use of X-rays.

The use of intraoperative imaging has the aim of providing additional information with regard to damaged tissues, of increasing the precision of the operative procedure, of reducing the intervention time in a context of mini-invasive surgery, which makes it possible to increase the clinical benefits.

Arthroscopy is a mini-invasive surgical intervention during which a miniature camera is introduced into a joint via a punctiform incision and a miniature instrument is introduced through a second punctiform orifice. The surgeon may thus carry out a diagnostic act while assessing the anatomical structures of the articular cavity and a suitable therapeutic act thanks to the appropriate instrument. This surgical approach, performed on an out-patient basis, has revolutionised surgical practice by making it possible to operate without opening up and with a decrease in post-operative morbidity (decrease in hospitalisation time and functional readaptation period).

The joints of the knee and the shoulder quickly benefited from this surgical innovation. All or part of the anatomical structures constituting these joints may be altered during a traumatism or an osteoarthritis. An arthroscopy of the knee may thus concern the meniscuses (for example in view of an ablation), the cartilage (for example in view of a regularisation), the synovial membrane (for example for an adhesion excision), the resection of small cartilaginous or bony fragments (foreign bodies), or certain more important surgeries of the knee (ligamentoplasty or others). Similarly, and in a non-exhaustive manner, an indication of arthroscopy of the shoulder may be posed for the care of rotator cuff calcific tendinopathy, a sub-acromial conflict treated by carrying out a bursectomy and acromioplasty under arthroscopy, or instead to qualify, intraoperatively, a rupture of the rotator cuff.

During an arthroscopy, the visual exploration of the inside of the articular cavity is limited to the surface of the structures constituting the joint, with, as consequences, potential diagnostic or therapeutic limitations.

An endoscopic ultrasound probe has the capacity of imaging several intra-articular structures (ligaments, cartilage) in depth and thus provides additional information compared to arthroscopy alone, which allows the surgeon to refine the diagnosis or even to adapt the operating procedure.

In order to be able to be used in the operating theatre, such a probe must be sterile. A medical device is considered to be sterile if the probability that a viable micro-organism is present is less than or equal to 10⁻⁶ (EN 556 standard). Sterilisation is a method aiming to make sterile the load to sterilise (EN 285 standard).

Conventional sterilisation methods include:
humid heat, called “saturated vapour” in an autoclave;
ionising radiation (y rays, electrons, UV);
plasma sterilisation using chemicals (for example hydrogen peroxide or peracetic acid).

The autoclave is the reference method in hospital settings; however, it is not suitable for an ultrasound probe because said probe is temperature sensitive.

This problem is thus an obstacle to the use of such a probe in clinical routine.

### DESCRIPTION OF THE INVENTION

An aim of the invention is to conceive an endoscopic ultrasound probe that is sterile, useable in any operating theatre, while procuring images of sufficient quality to make it possible to view anatomical structures in depth.

To this end, the invention proposes a sheath for an endoscopic ultrasound probe comprising:
a first portion suited to surrounding a catheter of the ultrasound probe, made of a material suited to allowing ultrasounds to pass, having a closed distal end,
a second portion suited to surrounding a handle and a cable for electrically connecting the probe to an ultrasound station, and
a fitting arranged between the first and the second portion, said fitting being suited to being removably attached to the handle.

In the present text, the relative terms “proximal” and “distal” are understood respectively to be a part of the probe situated on the side of the operator who handles it, and of a part of the probe on the side of the body of the patient into which it is intended to be inserted.

In a particularly preferred manner, the first portion is made of polyamide-polyether block copolymer.

In an advantageous manner, the first portion has a constant thickness.

According to an embodiment, the thickness of the first portion is comprised between 0.4 and 0.6 mm.

The second portion advantageously has a diameter greater than that of the first portion.

According to an embodiment, the fitting comprises a central opening for the passage of the catheter, the first portion being attached around said central opening of the fitting and the second portion being attached to a peripheral portion of the fitting.

According to an embodiment, the fitting is suited to being attached to the handle by a quarter turn type mechanism.

According to another embodiment, the fitting is suited to being attached to the handle by snap fitting.

The invention also relates to an endoscopic ultrasound probe, comprising:
an ultrasound sensor,
a connector suited to being connected to an ultrasound station,
a control handle,
an electrical connection cable extending between the connector and the handle,
a catheter comprising a proximal portion attached to the handle and a distal portion in which is arranged the ultrasound sensor,

said probe being characterised in that it comprises a sterilisable sheath such as described above, the first portion of the sheath surrounding the catheter, the second portion surrounding the handle and the cable, and the fitting being removably attached to the handle.

According to an embodiment, the ultrasound sensor is arranged facing a lens situated on a wall of the catheter.

The first portion of the sheath covers the portion of the catheter in which is arranged the ultrasound sensor.

According to an embodiment, the catheter has an inclined distal portion and/or a bevelled distal end. In this case, the lens is advantageously arranged at the distal end of the catheter.

In an alternative manner, the lens is arranged on the circumference of the catheter.

Another object of the invention is an ultrasound imaging system comprising an ultrasound station and an endoscopic ultrasound probe such as described above connected to said station by the connector, in which the station comprises a processor configured to process an image of an intra-articular zone of interest acquired by the probe and to determine, from said image, at least one of the following items of information:
a thickness of cartilage;
a rupture of the anterior cruciate ligament;
a location of an insertion point of the anterior cruciate ligament.

### DESCRIPTION OF THE FIGURES

Other characteristics and advantages of the invention will become clear from the detailed description that follows, with reference to the appended drawings in which:

FIG. 1 is a perspective view of a probe according to the invention;

FIG. 2 is a perspective view of a probe according to an embodiment in which the sheath is attached by clips on the handle;

FIG. 3 is a perspective view of the sheath of FIG. 2 before its attachment on the handle;

FIG. 4 is a perspective view of a probe according to an embodiment in which the sheath is attached by quarter turn on the handle;

FIG. 5 is a perspective view of the sheath of FIG. 4 before its attachment on the handle;

FIG. 6 is a schematic view of a first mounting for qualitatively testing the sheath;

FIGS. 7A-7D are ultrasound images obtained thanks to the mounting of FIG. 6, respectively in the absence of a sheath, with a sheath made of PEBD, with a sheath made of PEBAX™ 2533 and with a sheath made of PEBAX™ 3533;

FIG. 8 is a schematic view of a second mounting for qualitatively testing the sheath;

FIG. 9 is a graph representing the overall attenuation (in dB) calculated by integration of the attenuations of FIG. 9 over all of the frequencies for the different materials tested;

FIGS. 10A and 10B are ultrasound images of a thick animal cartilage obtained by a probe respectively exempt of a sheath and protected by a sheath made of PEBAX™ 2533;

FIGS. 11A and 11B are ultrasound images of a thin cartilage obtained by a probe respectively exempt of a sheath and protected by a sheath made of PEBAX™ 2533.

Identical reference signs from one figure to the other designate identical elements or elements fulfilling the same function.

### DETAILED DESCRIPTION OF EMBODIMENTS OF THE INVENTION

The endoscopic ultrasound probe (also called endo-ultrasound probe) forms part of an imaging system comprising an ultrasound station to which the probe is connected.

The station comprises an ultrasound control electronic, a processor suited to processing the images acquired by the probe, a viewing screen and a keyboard making it possible to enter data and to annotate the images.

During an operation under arthroscopy of the shoulder or the knee, the endo-ultrasound probe is inserted into the joint using the instrumental route. The probe is still used with the arthroscope which uses the other route. The surgeon brings the distal part of the probe into the zone of interest with the aid of the arthroscope and inspects the anatomical structures in this zone by orienting the probe towards them.

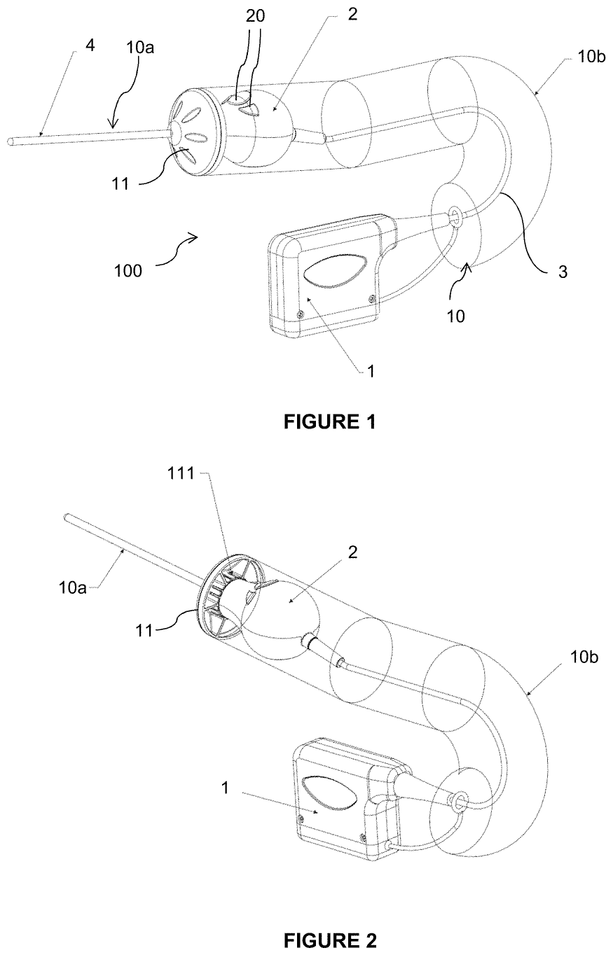

FIG. 1 is a perspective view of a probe according to the invention.

The probe 100 comprises a connector 1 through which it may be connected to the ultrasound station (which is not represented).

The connector 1 is connected to a control handle 2 through an electrical connection cable 3. The control handle is the member that the user holds in his hand during the intervention. The handle comprises one or more buttons 20 that the user can press for example to browse or to validate the acquisition of an image or a video.

From the handle 2 extends a catheter 4 in which is arranged an ultrasound sensor (not represented). The catheter has a length generally comprised between 10 and 20 cm and a diameter of several millimetres, for example 4 mm. The catheter may be made of stainless steel or titanium.

Although the catheter is represented straight in the appended figures, it may also have a curved portion and/or a bevelled distal end. “Curved portion” is taken to mean that the distal portion of the catheter is inclined with respect to the proximal portion of the catheter, said portions being connected by a curved portion. “Bevelled distal end” is taken to mean that the plane of the distal end is inclined with respect to the axis of revolution of the catheter. These inclinations are typically of the order of some ten to thirty degrees. Such a configuration of the catheter is particularly advantageous in arthroscopy, because it makes it possible to better adapt to the geometry of the joint to inspect. Thus, for example, the catheter may have a distal portion inclined by 10° with respect to the proximal portion of the catheter, and an end bevelled by an angle of 20° with respect to the axis of revolution of the catheter.

In an advantageous manner, the ultrasound sensor is situated facing a lens (not illustrated) arranged in the circumferential surface of the catheter, in the distal portion of the catheter. In an alternative manner, the lens may be arranged at the distal end of the catheter, notably when the catheter is curved or bevelled as mentioned above.

The sensor advantageously comprises a multi-element transducer arranged in the form of a linear array, for example 64 elements in number. The sensor has for example a length of 13 mm and the lens a length of 16.5 mm, the sensor being centred with respect to the lens.

The probe and the operation of the ultrasound station as such are known per se and will thus not be described in detail in the present text.

The probe is protected by a sterile sheath 10, which covers it entirely, from the distal end of the catheter up to the connector. The sheath is in one piece and only has an opening at the level of the connector 1.

The sheath has a first part 10a suited to surrounding the catheter, and a second part 10b, wider, surrounding the handle and the connection cable. The dimensions of the sheath are chosen to adapt to the shape and to the dimensions of the probe. Generally speaking, the first and the second part each have a cylindrical shape of circular section.

The first part 10a hugs the shape of the catheter 4 as closely as possible in order not to increase excessively the bulk of the catheter, a slight clearance being arranged to facilitate the slipping of the sheath onto the catheter. For example, for a sheath of 0.5 mm thickness and a catheter of 4 mm diameter, the outer diameter of the first portion is of the order of 5.4 mm. The first portion 10a of the sheath is closed at its distal end, including facing the lens. The first portion 10a is sufficiently flexible given its material and its thickness to adapt to the shape of the catheter, whether it is straight or curved.

The first and the second part of the sheath are connected by a fitting 11 thanks to which the sheath may be removably attached to the handle of the probe.

The fitting is typically made of a thermoplastic material.

The first portion 10a of the sheath is made of a material suited to allow the ultrasound signal to pass with a sufficiently low attenuation so that imaging can be carried out with sufficient resolution. The choice of a material suited for this function will be described in detail hereafter. Preferably, the first portion of the sheath is made of a polyamide-polyether block copolymer sold notably under the denomination PEBAX™, or made of PEBD. Preferably, PEBAX™ 2533 is preferred, PEBAX™ 3533 being satisfactory but less preferred. The thickness of the first portion of the sheath is chosen sufficiently thin so as not to bring about a too important attenuation of the ultrasounds. For example, the thickness of this first portion is comprised between 0.4 and 0.6 mm, in a preferred manner 0.5 mm. This thickness is advantageously constant over the entire length of the first portion.

The second portion 10b of the sheath may be made of the same material as the first portion or made of another material, this second portion not being intended to allow ultrasounds to pass. The second portion does not necessarily comprise means of attachment to the connector. It is in fact sufficiently long to receive the probe up to the non-sterile zone of the operating site, even if it is simply slipped around the connection cable.

The fitting 11 has a central opening for the passage of the catheter 4, around which it extends radially, the first portion 10a of the sheath being attached to a central portion of the fitting, around said opening 110, and extends from the distal end of the fitting; the second portion 10b of the sheath is attached to a peripheral portion of the fitting and extends from the proximal end of the fitting. The first and the second portions 10a, 10b are attached in a leak tight manner to the fitting 11 by any appropriate means, for example by bonding on the fitting or by over-moulding of the fitting.

The fitting 11 and the handle 2 advantageously have mutual reversible fastening together means. According to an embodiment, the fastening together is carried out by a quarter turn mechanism. In an alternative manner, the fastening together may be carried out by snap fitting.

The material(s) forming the two portions of the sheath and the fitting may be sterilised by ionising radiation without degrading. Preferably, the sterilisation is carried out by exposure to ethylene oxide followed by exposure to y rays.

The sheath 10 being removable vis-à-vis the remainder of the probe and entirely covering the part of the probe situated in the operating field, it may be sterilised in isolation and makes it possible to procure a sterile endoscopic ultrasound device without risking damaging the functional components of the probe. Typically, the sheath is sterilised before being packaged in a packaging suited to preserving the sterility and is only extracted from this packaging to be put in place on the probe.

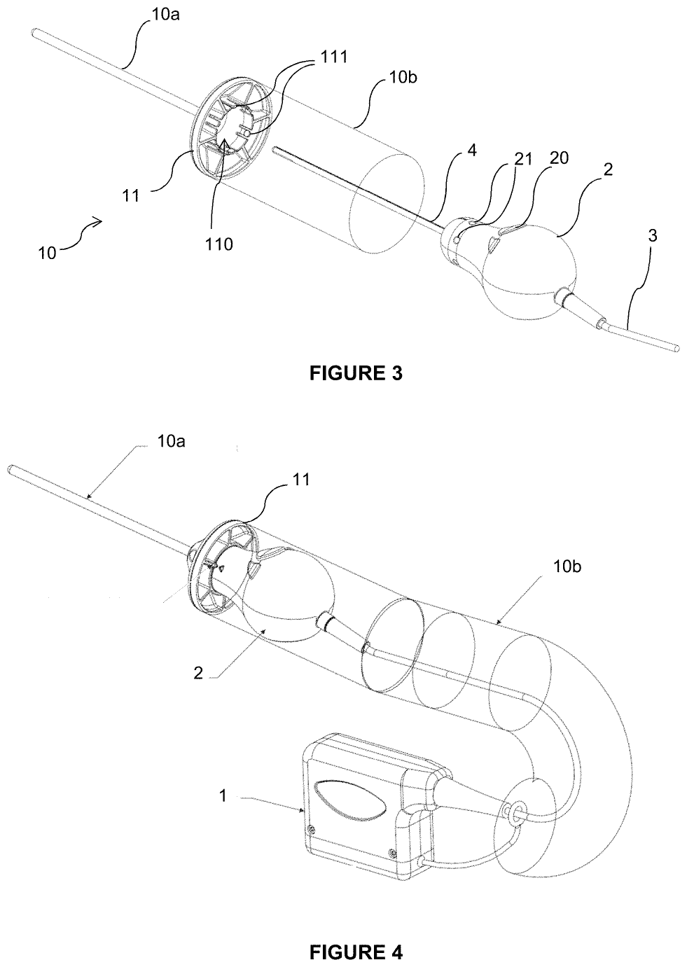

FIGS. 2 and 3 illustrate a first embodiment of the probe, respectively after and before fastening the sheath on the handle.

In this embodiment, the attachment is ensured by snap fitting. To this end, the fitting 11 comprises clips 111 which extend from its proximal surface, for example spread out at regular intervals on a circle concentric to the axis of the catheter, and the handle 2 comprises on its outer surface corresponding reinforcements 21. After assembly of the first portion of the sheath on the catheter, the clips 111 engage in the corresponding reinforcements 21 and thus maintain the fitting 11 on the handle 2.

Naturally, the shape of the fitting, the handle and the respective attachment means could vary without however going beyond the scope of the present invention.

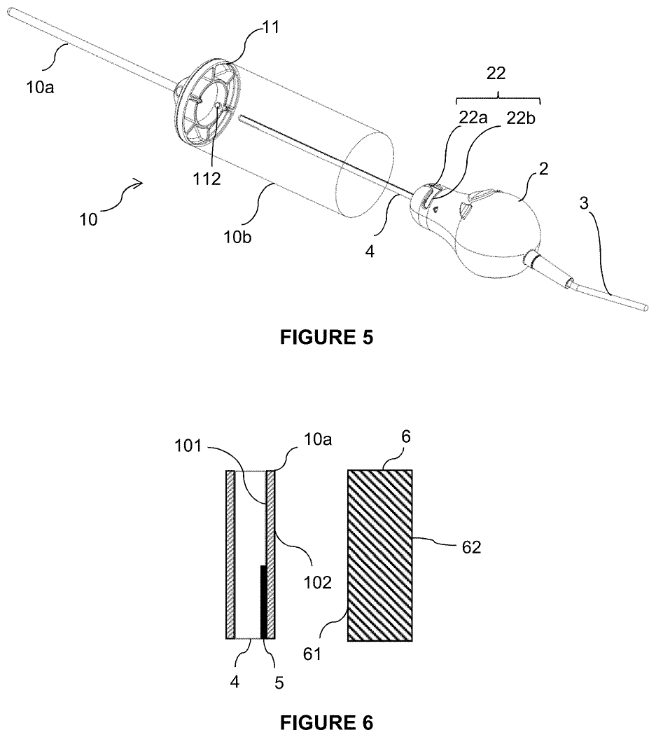

FIGS. 4 and 5 illustrate a second embodiment of the probe, respectively after and before fastening the sheath on the handle.

In this embodiment, the attachment is ensured by a quarter turn mechanism. To this end, the fitting 11 comprises two lugs 112 radially projecting inwards, preferably radially opposite, and the handle 2 comprises two corresponding grooves arranged in its outer surface. Each groove 22 comprises an axial inlet portion 22a and a maintaining portion 22b extending circumferentially from the proximal end of the inlet portion 22a. After assembly of the first portion 10a of the sheath on the catheter 4, the lugs 112 engage together in the axial inlet portions 22a of the grooves until reaching full travel at their proximal end, then in the maintaining portions 22b under the effect of a relative pivoting of the handle and the fitting.

These two embodiments have been described because they constitute a particularly simple and sure means for removably attaching the fitting on the handle. However, those skilled in the art will be able to choose any other suitable reversible fastening together means (for example screwing, friction, etc.) without however going beyond the scope of the present invention.

The modalities for choosing the material of the first portion of the sheath will now be described.

The inventors have tested different materials transparent to ultrasonic waves and have qualitatively and quantitatively evaluated them to verify their capacity to allow the ultrasound signal to pass with a sufficiently low attenuation.

FIG. 6 is a schematic diagram of a first mounting for a qualitative test.

The ultrasound sensor 5 of the probe is arranged facing a wall 6 made of PVC, the assembly being immersed in water.

A first ultrasound image is acquired without the sheath, then a sheath 10a constituted of each of the materials to test is slipped onto the catheter 4, and a respective ultrasound image is acquired.

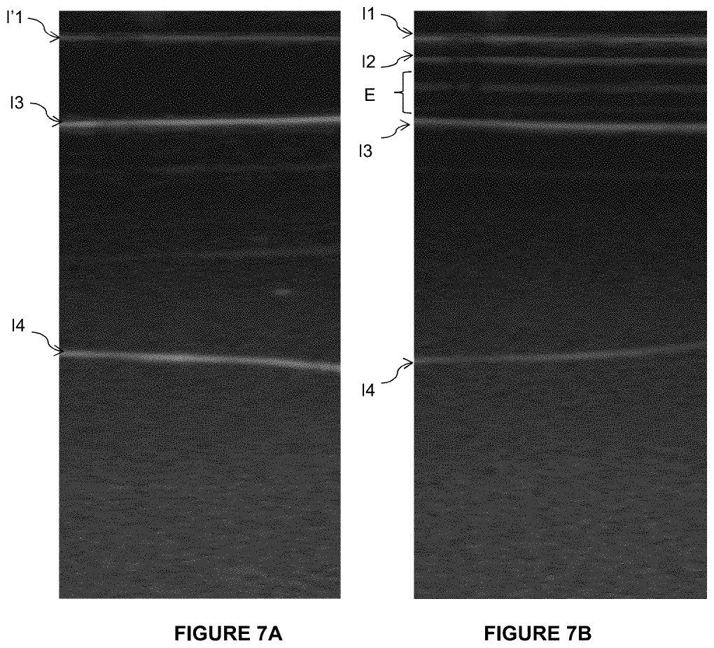

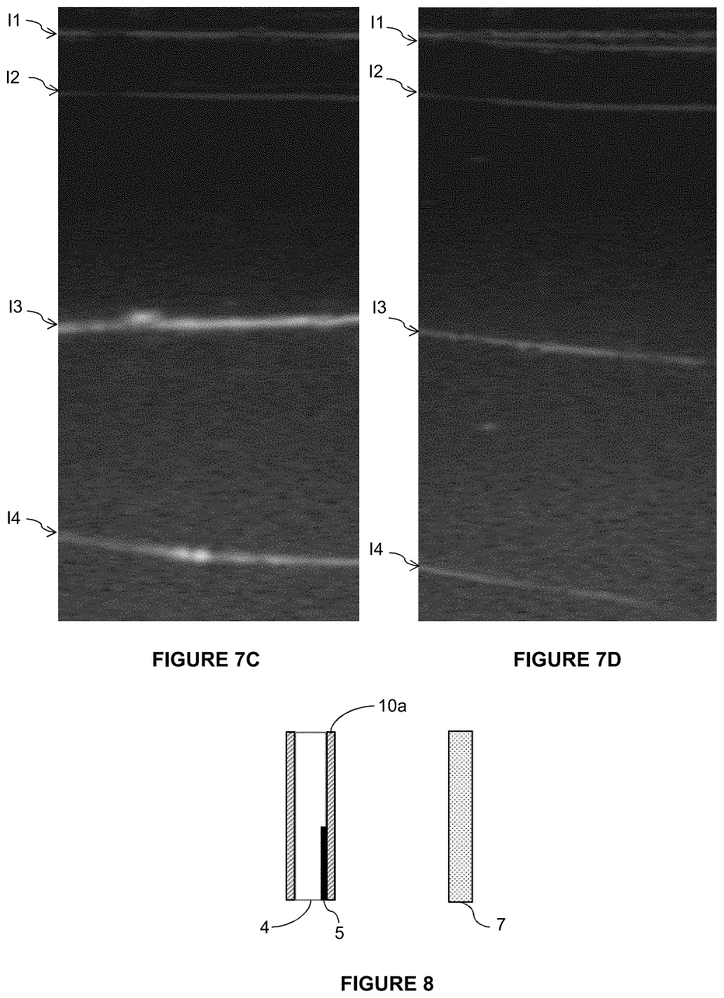

The results presented in FIGS. 7A-7D are respectively obtained without a sheath, with a sheath made of PEBD, with a sheath made of PEBAX™ 2533 (preferred material) and made of PEBAX™ 3533. The white lines designated by 11, 12, 13 and 14 correspond respectively to the interface between the lens and the sheath, the interface between the sheath and water, the interface between water and PVC (surface of the wall facing the probe) and the interface between PVC (surface of the wall opposite to the probe) and water. In the case of FIG. 7A, in the absence of a sheath, the interface between the lens and water is designated by I′1.

A first criterion for qualification of a material envisaged for the sheath is the visibility of these interfaces. It is sought to obtain a visibility as close as possible to the visibility obtained without a sheath. The loss of visibility of the interfaces 61, 62 of the PVC wall is due to the absorption of ultrasounds in the sheath and to the energy reflected by the inner 101 and outer 102 surfaces of the sheath.

Furthermore, the potential lines designated by E correspond to parasitic interfaces due to a cavity phenomenon in the thickness of the sheath. This phenomenon is caused by a difference in acoustic impedance between the sheath and water. Hence, the ultrasounds travel back and forth multiple times between the outer surface of the sheath and the probe, each back and forth journey producing an echo which is translated by a horizontal line on the image. Such parasitic interfaces adversely affect the quality of the image. These parasitic echoes are all the more problematic given that in the targeted intra-articular application the probe must be very close to the tissues to ultrasound, or even in contact therewith, which implies that potential parasitic echoes are superimposed on the anatomical structures to observe.

The presence and the number of parasitic echoes thus constitutes a second criterion for qualification of the material. Such echoes must be avoided or at least minimised.

It may be observed in these images that the sheath made of PEBD produces parasitic echoes, even though the expected interfaces have good visibility. The sheath made of PEBAX™ 2533, which is the most high-performance material, does not generate parasitic echoes while procuring clearly visible interfaces. PEBAX™ 3533 also appears as a relatively satisfactory material.

The table below recapitulates the tested materials and their classification in terms of number of parasitic echoes and visibility of interfaces.

| | Number of | Visibility of |
|---|---|---|
| Sheath material | parasitic echoes | the interfaces |
| Without sheath | 0 | +++ |
| PA12 | 4 | + |
| PEBD | 2 | ++ |
| PVC | 2 | + |
| Silicone | 0 | − |
| TPU95 | 2 | −− |
| TPU98 | 3 | −− |
| PEBAX ™ 2533 | 0 | ++ |
| PEBAX ™ 3533 | 0 | + |
| PEBAX ™ 4533 | 0 | − |

Another study was conducted to evaluate quantitatively the attenuation caused by the different materials envisaged for the sheath.

FIG. 8 is a schematic diagram of a second mounting for a qualitative test.

The ultrasound sensor 5 of the probe is arranged facing a wall 7 made of metal, which is a good reflector of ultrasounds, the assembly being attached by a bench to maintain a constant distance between the probe and the metal wall.

A first ultrasound image is acquired without the sheath, then a sheath 10a constituted of each of the materials to test is slipped onto the catheter 4, and a respective ultrasound image is acquired. An attenuation calculation is then performed.

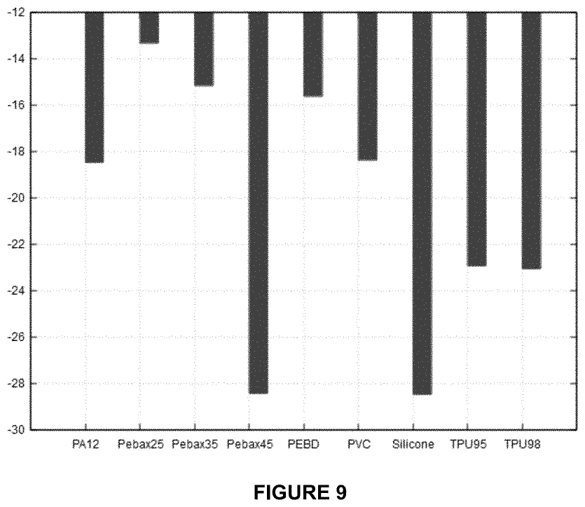

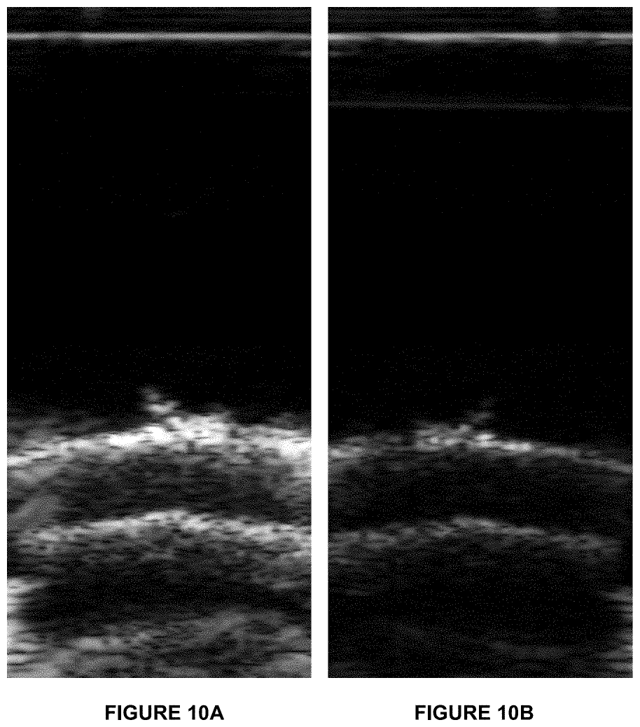

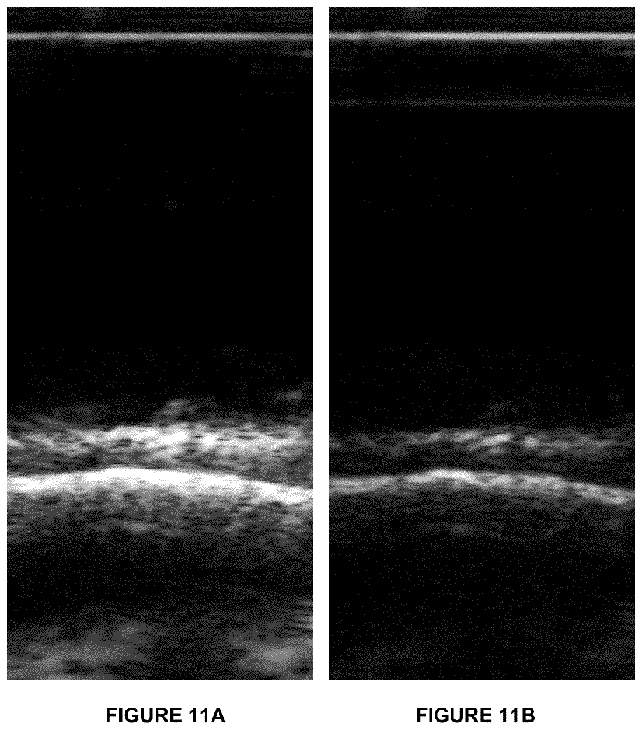

FIG. 9 is a graph representing the overall attenuation (that is to say integrated over the whole of the frequency range of interest, which is comprised between 10 and 15 MHz) (in dB) for the different materials tested.

It may be observed that silicone and PEBAX™ 4533 have strong absorption, and are thus not preferred, even though they do not generate parasitic echoes. PEBAX™ 2533 is the best candidate, with an absorption of around 13 dB, followed by PEBAX™ 3533 (15 dB) and PEBD (15.5 dB). However, PEBD will not be retained on account of the parasitic echoes that it generates.

In conclusion, PEBAX™ 2533 is the tested material that has the least attenuation. In addition, it is important to note that the sheaths made of PEBAX™ tested were thicker (around 2 mm thickness) than the others (around 0.8 mm). Thus, in conditions of identical thickness, a sheath made of PEBAX™ 2533 would have even less attenuation than the others.

To validate that PEBAX™ 2533 indeed corresponds to the expectations, cartilage images were taken while maintaining the probe fixed on animal anatomical parts. All the images were recorded with the same adjustments of parameters. Thick (that is to say of around 2.6 mm thickness) (cf. FIGS. 14A-14B) and thin (that is to say of around 1.6 mm thickness) (cf. FIGS. 15A-15B) cartilage images were recorded (visible in the lower part of the images). The interfaces delimiting the cartilage are still visible with the sheath made of PEBAX™ 2533.

When the probe is bare in water, the interfaces are very shiny and saturate the image: it would be necessary to decrease the gain to observe it correctly. With the sheath made of PEBAX™ 2533, the saturation disappears, as if the gain had been decreased to an acceptable level.

From an acoustic viewpoint, PEBAX™ 2533 thus appears as the best material: its acoustic impedance is close to that of water (no parasitic echoes due to the cavity phenomenon) and it absorbs little ultrasounds. Consequently, the images obtained through this sheath have sufficient quality for a viewing in depth of the anatomical structures of interest, while ensuring the sterility of the probe.

In addition, PEBAX™ 2533 is a translucid material, through which the inside of the probe may be seen, notably the position of the ultrasound sensor on the arthroscopic images, which makes it possible to facilitate the use of the probe by a surgeon.

Thanks to said probe, endoscopic ultrasound imaging makes it possible in particular:
to see tissue lesions (partial ligament rupture, intratendinous calcification, others) not flush with the surface, consequently with a better localisation;
to identify, using new parameters, pathological tissular reworkings/alterations, as has already been demonstrated, for example, within the context of osteoarthritic disease (modifications of the echogenicity properties of the cartilage, at the interfaces);
to estimate the thickness of the cartilage from the correlation between the time of flight of the ultrasounds and the actual thickness measured by MRI of the cartilage;
to facilitate the carrying out of arthroscopic calcification resection procedures (visible by ultrasounds, but not with the naked eye), tenotomy or tenodesis following better ultrasonic estimation of the inflammatory state of the tendon, etc.

The invention has a particular interest in the treatment of lesions of the cartilage and of the anterior cruciate ligament (hereafter designated by the acronym ACL) in the knee.

In the knee, the hyaline cartilage is not vascularised and the cells are nourished by the synovial liquid which enables the passage of nutrients through the cartilage. Thus, mobilisation of the joint is necessary to avoid suffering and alteration of the cartilage. In addition, the cartilage does not restore itself and its healing generates an irregular fibrous tissue which does not procure any biomechanical quality. In the event of lesion, this healing then leads to secondary osteoarthritis. Primitive osteoarthritis is for its part due to natural wear of the cartilage.

The probe enables an intraoperative evaluation of lesions of the cartilage of the knee to treat. It enables a cleaning of the lesion in order to determine very precisely the dimensioning, in surface area and in depth, of the surface to treat and thus allows the surgeon a therapeutic choice suited to each of his patients. Furthermore, the precision of its technology offers the possibility of differentiating the fibrocartilage from the hyaline cartilage, very desirable for the evaluation of cellular therapy products.

For gonarthrosis pathologies, the evaluation of the state of the cartilage and notably of early osteoarthritis associated with a meniscal lesion for example may make it possible to adapt the resumption of physical activities with a view to protecting the cartilage.

The positioning of the femoral tunnel during partial reconstructions of the ACL is crucial: incorrect positioning may lead to the onset of symptoms of anterior or rotational instability or to loss of function. Reference anatomical structures such as the “resident's ridge” make it possible to pinpoint the placement of the tunnel: the endo-ultrasounds makes it possible to view this enthesis of the ACL.

The invention also has a particular interest in the treatment of calcifying tendinitis of the shoulder.

The literature shows that the association of a complete cleaning of the calcifications combined with a preservation of the tendon optimises the postoperative recoveries of patients.

The use of the intra-articular ultrasound probe makes it possible to better locate calcifications while avoiding the trituration procedure. The probe offers more precision in the surgical procedure by pinpointing calcifications situated inside the tendon itself: it pinpoints calcification and sends back an image during the detection of the calcifying lesion.

1. A sheath for an endoscopic ultrasound probe, comprising:
a first flexible portion configured for surrounding a catheter of the ultrasound probe, made of a material suited to allowing ultrasounds to pass, having a closed distal end,
a second portion configured for surrounding a handle and a cable for electrically connecting the probe to an ultrasound station, and
a fitting arranged between the first and the second portion, said fitting being configured for being removably attached to the handle.

2. The sheath of claim 1, wherein the first portion is made of a polyamide-polyether block copolymer.

3. The sheath of claim 1, wherein the first portion has a constant thickness.

4. The sheath of claim 1, wherein the thickness of the first portion is comprised between 0.4 and 0.6 mm.

5. The sheath of claim 1, wherein the second portion has a diameter greater than a diameter of the first portion.

6. The sheath of claim 1, wherein the fitting comprises a central opening for the passage of the catheter, the first portion being attached around said central opening of the fitting and the second portion being attached to a peripheral portion of the fitting.

7. The sheath of claim 1, wherein the fitting is configured for being attached to the handle by a quarter turn type mechanism.

8. The sheath of claim 1, wherein the fitting is configured for being attached to the handle by snap fitting.

9. An endoscopic ultrasound probe, comprising:
an ultrasound sensor,
a connector configured for being connected to an ultrasound station,
a control handle,
an electrical connection cable extending between the connector and the handle,
a catheter comprising a proximal portion attached to the handle and a distal portion in which is arranged the ultrasound sensor, and
a sheath comprising:
a first flexible portion configured for surrounding a catheter of the ultrasound probe, made of a material suited to allowing ultrasounds to pass, having a closed distal end,
a second portion configured for surrounding a handle and a cable for electrically connecting the probe to an ultrasound station, and
a fitting arranged between the first and the second portion, said fitting being configured for being removably attached to the handle,
wherein the first portion of the sheath surroundings the catheter, the second portion surrounds the handle and the cable, and the fitting is removably attached to the handle.

10. The probe of claim 9, wherein the ultrasound sensor is arranged facing a lens situated on a wall of the catheter.

11. The probe of claim 10, wherein the first portion of the sheath covers the portion of the catheter in which is arranged the ultrasound sensor.

12. The probe of claim 9, wherein the catheter has a curved distal portion and/or a bevelled distal end.

13. The probe of claim 1Z wherein the ultrasound sensor is arranged facing a lens located at the distal end of the catheter.

14. The probe of claim 10, wherein the lens is arranged on the circumference of the catheter.

15. An imaging system comprising an ultrasound station and an endoscopic ultrasound probe according to claim 9 connected to said ultrasound station by the connector, wherein the ultrasound station comprises a processor configured to process an image of an intra-articular zone of interest acquired by the probe and to determine, from said image, at least one of the following items of information:
a cartilage thickness;
a rupture of the anterior cruciate ligament; and
an emplacement of an insertion point of the anterior cruciate ligament.
